(19) United States
(12) Patent Application Publication
(10) Pub. No.: US 2023/0022128 A1
(43) Pub. Date: Jan. 26, 2023

(54) OZONE TREATMENT DEVICE

(71) Applicant: Preservation Tech LLC, Hull, IA (US)

(72) Inventors: Kevin York, Hull, IA (US); Sannel Patel, Hull, IA (US)

(21) Appl. No.: 17/866,211

(22) Filed: Jul. 15, 2022

Related U.S. Application Data
(60) Provisional application No. 63/222,366, filed on Jul. 15, 2021.

(51) Int. Cl. A61L 2/20; C01B 13/10; A61L 2/24

(52) U.S. Cl. CPC A61L 2/202; C01B 13/10; A61L 2/24; A61L 2202/25; A61L 2202/11; A61L 2202/14; A61L 2202/13; A61L 2202/16

(57) ABSTRACT

An ozone treatment device includes an ozone destruct module and an ozone generator mounted within a housing. The ozone destruct module and the ozone generator are mounted between corresponding air inlets and air outlets formed in the housing. The ozone treatment device also includes a controller mounted to the housing. The controller is configured to generate ozone with the ozone generator for a first time period and to automatically destruct ozone with the ozone destruct module for a second time period following the first time period. In certain implementations the treatment device includes an input device for receiving an operator input. In such cases the controller can determine the first and second time periods based on the operator input.

### CROSS-REFERENCE TO RELATED APPLICATION(S)

This application claims the benefit under 35 U.S.C. § 119(e) to U.S. Provisional Application 63/222,366, filed Jul. 15, 2021, and entitled “Ozone Treatment Device,” which is hereby incorporated herein by reference in its entirety.

### TECHNICAL FIELD

The disclosed technology relates generally to ozone generation and destruction.

### BACKGROUND

Ozone has long been used as a broad-spectrum biocide against microorganisms. The triatomic form of ozone and its ability to oxidize microorganisms has been applied as a disinfectant in many settings, including, for example, to treat water, kill food bacteria, clean laundry and other fabrics, decontaminate hospital operating rooms, and deodorize air and objects after, for example, a fire.

Ozone is made by adding energy to oxygen molecules (O₂), which causes oxygen atoms to split apart. In some cases, these single oxygen atoms temporarily combine with other oxygen molecules to create ozone molecules (O₃). This process naturally occurs in response to lightning and sun light. Ozone can be artificially produced through the same mechanisms, by splitting diatomic oxygen molecules with energy from corona discharge or ultraviolet light. Ozone generators have typically incorporated one of these techniques to produce ozone that can be used as a disinfectant for both small- and large-scale applications.

Ozone tends to naturally decay over time, changing from triatomic oxygen molecules to diatomic oxygen molecules. After disinfecting a room with ozone, the ozone will eventually dissipate as it changes to diatomic oxygen. Once the ozone level has reduced to a safe threshold, people and animals can safely enter the now-disinfected space. In some cases, especially when there is a high or unsafe level of ozone present, an ozone destruct unit may be used to remove ozone from the room more quickly.

There is a need in the art to improve methods and devices for generating and destructing ozone.

### SUMMARY

This disclosure generally relates to generating ozone to treat an enclosed space and destructing ozone within an enclosed space. Various aspects of the disclosure relate to ozone treatment devices that include both an ozone generator and an ozone destruct module, as well as methods for operating ozone treatment devices.

An ozone treatment device is provided according to one aspect of the disclosure. The ozone treatment device includes an ozone destruct module and an ozone generator mounted within a common housing. The housing has a first air inlet, a second air inlet, a first air outlet, and a second air outlet. The treatment device also includes a controller mounted to the housing. The ozone destruct module is coupled to the controller and mounted within the housing between the first air inlet and the first air outlet. The ozone generator is also coupled to the controller and mounted within the housing between the second air inlet and second air outlet. The controller is configured to operate the ozone generator for a first time period. The controller is also configured to automatically operate the ozone destruct module for a second time period following the first time period.

Various implementations of the ozone treatment device include one or more of the following features and/or elements. In some cases the ozone treatment device further includes an input device coupled to the controller. The controller is further configured to receive an operator input from the input device and to determine the first time period based on the operator input. In some cases the operator input is a treatment time and/or a size of a room. In various implementations the controller is configured to determine the second time period based on the operator input. In some cases the ozone destruct module includes destruct media and a fan, and the controller is further configured to determine the second time period based on a reduction rate of the destruct media and a speed of the fan.

In various implementations the ozone treatment device further includes an ozone sensor. In some cases the ozone generator comprises a support plate and the support plate is mounted in the housing above the ozone destruct module. In some cases the support plate substantially seals off the ozone generator from the ozone destruct module. In various implementations the housing comprises a portable case with a handle for hand-carrying. In various implementations the housing comprises a rollable housing with wheels.

Another aspect of the disclosure relates to an ozone treatment device that includes a housing, an ozone generator, an ozone destruct module, an ozone sensor, and a controller coupled to the ozone generator, the ozone destruct module, and the ozone sensor. The housing includes an ozone generation flow path and an ozone destruct flow path substantially sealed off from the ozone generation flow path. The ozone generation flow path and the ozone destruct flow path are vertically stacked in the housing, one over the other. The ozone generator is positioned in the ozone generation flow path and the ozone destruct module is positioned in the ozone destruct flow path. The controller is configured to generate ozone with the ozone generator for a first time period and then automatically destruct ozone with the ozone destruct module for a second time period following the first time period.

Various implementations of the ozone treatment device include one or more of the following features and/or elements. In some cases the ozone treatment device also includes an input device coupled to the controller. The controller is further configured to receive an operator input from the input device and determine the first time period and the second time period based on the operator input. In various implementations the controller is configured to determine an ozone concentration level using the ozone sensor following the first time period. The controller is configured to generate ozone with the ozone generator for an additional time period if the ozone concentration level does not meet a concentration threshold level. In some cases the controller is configured to determine an ozone concentration level using the ozone sensor following the second time period. The controller is configured to destruct ozone with the ozone destruct module for an additional time period if the ozone concentration level does not meet a concentration threshold level.

A method for operating an ozone treatment device is provided according to another aspect of the disclosure. The method includes generating ozone with an ozone treatment device for a first time period. The ozone treatment device includes an ozone generator, an ozone destruct module, and a controller coupled to the ozone generator and the ozone destruct module. The method also includes automatically destructing ozone with the ozone treatment device for a second time period following the first time period.

Various implementations of the method for operating an ozone treatment device include one or more of the following features and/or steps. In some cases the method includes receiving an operator input with an input device of the ozone treatment device and determining, with the controller, the first time period and the second time period based on the operator input. In some cases the operator input is a room size. In some cases the method also includes determining, with the controller, an ozone concentration level sensed by an ozone sensor of the ozone treatment device. The method includes generating ozone with the ozone treatment device for an additional time period if the ozone concentration level does not meet a concentration threshold level. In various cases the method includes determining, with the controller, an ozone concentration level sensed by an ozone sensor of the ozone treatment device. The method includes destructing ozone with the ozone destruct module for an additional time period if the ozone concentration level does not meet a concentration threshold level.

While multiple embodiments are disclosed, still other embodiments will become apparent to those skilled in the art from the following detailed description, which shows and describes illustrative embodiments. As will be realized, the various implementations are capable of modifications in various obvious aspects, all without departing from the spirit and scope thereof. Accordingly, the drawings and detailed description are to be regarded as illustrative in nature and not restrictive.

### BRIEF DESCRIPTION OF THE DRAWINGS

FIG. 1 is a front perspective view of an ozone treatment device according to some embodiments.

FIG. 2 is a rear perspective view of the ozone treatment device of FIG. 1.

FIG. 3 is a perspective view of a housing of the ozone treatment device of FIG. 1 with fan filters according to some embodiments.

FIG. 4 is a perspective view of an ozone destruct module according to some embodiments.

FIG. 5 is an exploded, perspective view of the ozone destruct module of FIG. 4.

FIG. 6 is a perspective view of an ozone destruct subassembly according to some embodiments.

FIG. 7 is a perspective view of an ozone generator according to some embodiments.

FIG. 8 is a side cross-sectional view of the ozone generator of FIG. 7.

FIG. 9 is an exploded perspective view of the ozone generator of FIG. 7.

FIG. 10 is a perspective view illustrating the mounting of the ozone destruct module of FIG. 4 and the ozone generator of FIG. 7 within the housing of FIG. 3 according to some embodiments.

FIG. 11 is a perspective cross-sectional view of an ozone treatment device according to some embodiments.

FIG. 12 is an exploded perspective view of a control panel for an ozone treatment device according to some embodiments.

FIG. 13 is an exploded perspective view showing the mounting of the control panel of FIG. 12 to the housing of FIG. 3 according to some embodiments.

### DETAILED DESCRIPTION

This disclosure generally describes embodiments that relate to generating ozone and destructing ozone. According to one aspect of the disclosure, an ozone treatment device includes an ozone generator and an ozone destruct module. The treatment device is configured to generate ozone in an enclosed space to, for example, sanitize or disinfect the space. After generating the ozone, the ozone treatment device can be used again to destruct the ozone in the enclosed space. In certain implementations, the ozone treatment device is configured to destruct the ozone within the enclosed space more quickly than the ozone would naturally decay within the space.

Certain implementations of ozone treatment devices described herein thus provide a convenient and effective way to both disinfect an enclosed space and to more quickly remove ozone after disinfection. Implementations of the disclosed ozone treatment devices can be useful for sanitizing and disinfecting areas exposed to human-borne and animal-borne pathogens, and in certain cases can be especially useful for cleaning high-traffic areas and enclosed spaces that see frequent occupant turnover.

According to another aspect of the disclosure, implementations of an ozone treatment device are configured to generate ozone for a first time period and then automatically destruct ozone for a second time period following the first time period. According to this aspect, the ozone treatment device includes a controller operably coupled to an ozone destruct module and an ozone generator. The controller is configured to generate ozone with the ozone generator for the first time period and then automatically destruct ozone with the ozone destruct module for the second time period.

In certain implementations, the ozone treatment device has an input device operably coupled to the controller. In these cases, the controller is configured to receive an operator input from the input device and determine the first time period based on the operator input. As just one example, an operator may enter a desired treatment time that is the first time period. As another example, an operator may enter the size of a room with the input device and the controller can determine the first time period based on the room size, a desired ozone concentration, an ozone generation rate, and/or other factors. The controller may also be configured to determine the second time period based on the operator input and possibly other factors such as, for example, the reduction rate of the destruct media.

According to another aspect of the disclosure, implementations of the disclosed technology include an ozone destruct module and an ozone generator mounted within a common housing to form an ozone treatment device capable of both generating and destructing ozone. In these implementations, the device has an ozone generation flow path extending between an air inlet and an air outlet formed in the housing. The device also includes an ozone destruct flow path extending between another air inlet and another air outlet formed in the housing. According to this aspect, the housing is formed so that the ozone generation flow path is substantially sealed off from the ozone destruct module. In certain implementations the ozone generation flow path is located in a top portion of the housing overtop the ozone destruct flow path. In some cases components of the ozone generation flow path are mounted to a support or mounting plate, or within a tubular housing, positioned above the ozone destruct flow path. In these types of configurations the support plate and/or tubular housing can effectively separate, and in some cases seal off the generation flow path from the destruct flow path.

According to another aspect of the technology disclosed herein, certain implementations provide an easily transportable form factor for the ozone treatment device. In some cases the ozone treatment device has an ozone generator and an ozone destruct module both mounted in a housing that includes a portable case. The case may have a handle for carrying the ozone treatment device and/or casters/wheels and a handle for rolling the ozone treatment device.

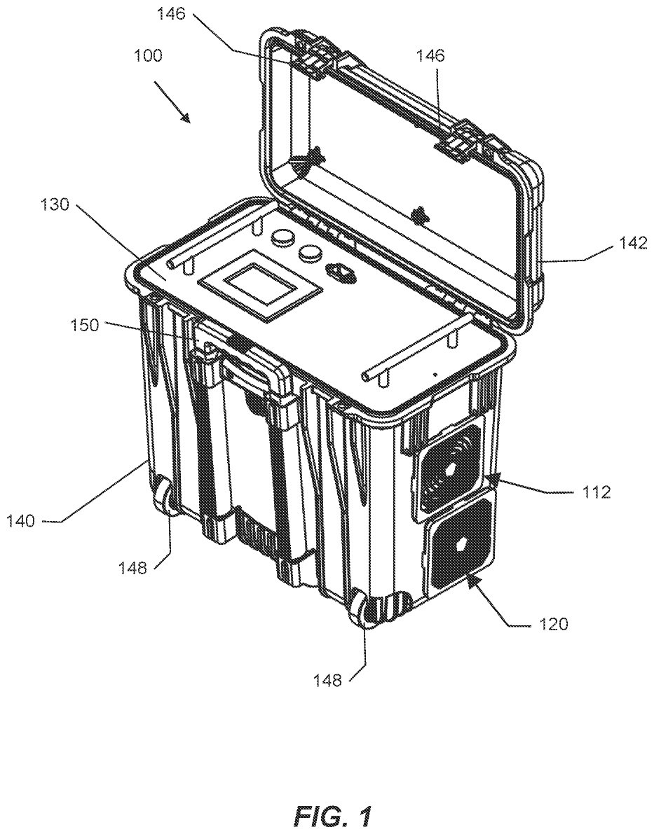

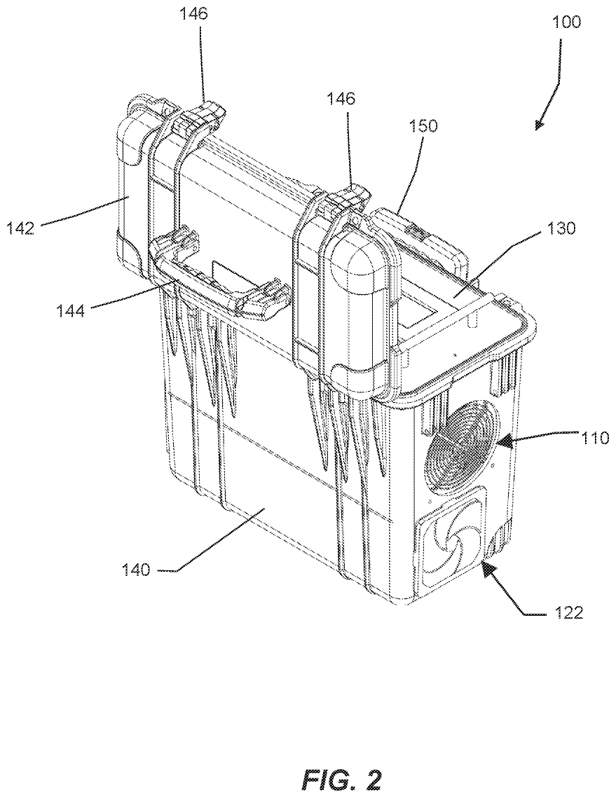

Turning to the drawings, FIG. 1 is a front perspective view of an ozone treatment device 100 according to some implementations. FIG. 2 is a rear perspective view of the treatment device 100. During ozone generation, ambient air enters an ozone generation flow path through an ozone generation inlet 110 and exits the treatment device through an ozone generation outlet 112, containing more ozone than before. For ozone destruction, ambient air containing ozone enters an ozone destruct flow path through an ozone destruct inlet 120 and exits through an ozone destruct outlet 122 containing less ozone than when entering the treatment device 100. The device includes a control panel 130 configured to control operation of the ozone generation and destruct flow paths.

Ozone treatment devices according to the disclosed technology include a housing that can take on a number of forms. In various implementations the housing is portable, though it will be appreciated that a wide variety of configurations can be used. As shown in FIGS. 1-2, in various implementations the ozone treatment device 100 has a housing 140 that is a durable, portable case including a hinged lid 142 and a handle 144 for carrying the device by hand. The lid includes latches 146 for securing the lid when closed. In this example the housing also includes wheels 148 and a telescoping pull handle 150 that enable a person to pull the device along in a rolling fashion. The portable nature of the housing 140 provides a convenient and easily transportable form-factor for the ozone treatment device 100. In certain implementations the portable configuration can, for example, make it easier to disinfect multiple enclosed spaces with a single ozone treatment device.

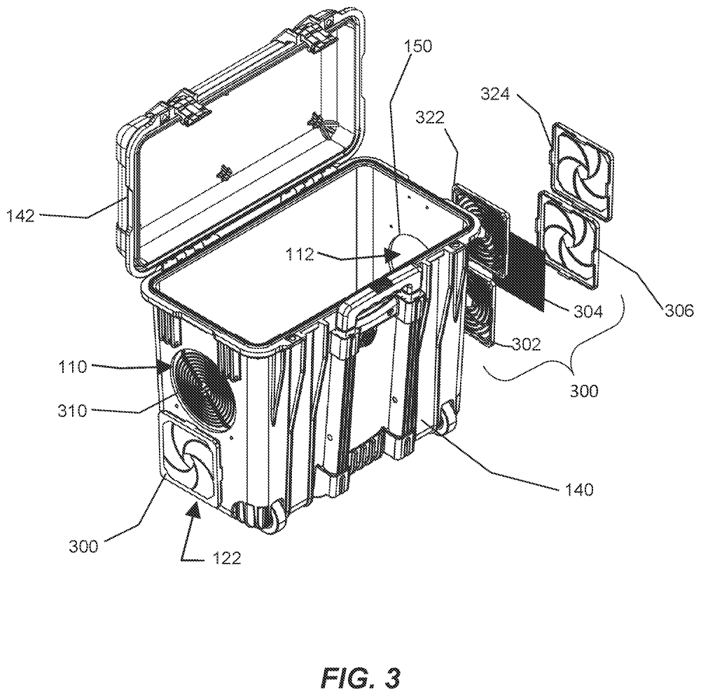

FIG. 3 is a perspective view of the device housing 140 without the internal components. The housing 140 includes openings 150 that provide the ozone generation and destruct inlets and outlets. According to various implementations, the housing 140 includes fan filters and/or fan guards mounted to one or more of the openings to, for example, filter incoming or outgoing air and prevent a person's fingers from touching an operating fan. In the illustrated implementation, a fan filter 300 is mounted over both the ozone destruct inlet 120 (shown in FIG. 1) and ozone destruct outlet 122 (shown in FIGS. 2 and 3) at the beginning and end, respectively, of the ozone destruct flow path. The fan filter 300 in this example includes a filter guard 302, a filter media panel 304 and a filter retaining member 306, shown in the exploded portion of FIG. 3. The filter retaining member 306 mounts to the filter guard and retains the filter media panel 304 in place. In some cases the filter media panel 304 is a fiber dust filter that limits the flow of dust into and out of the ozone treatment device.

Turning to the ozone generation flow path, in this case a metal fan guard 310 is mounted over the ozone generation inlet 110. In some cases the use of a fan guard without a filter media panel can increase the airflow into the ozone generation path. The ozone generation outlet 112 in this implementation has a fan filter that includes a filter guard 322 and a filter retaining member 324. In this and various other implementations, a filter media panel is not used for the ozone generation outlet 112. In some cases this omission of the filter media panel can lead to less maintenance and replacement of filter panels since the flow of ozone from the generation path can more quickly break down the media than would otherwise occur. It should be appreciated, though, that according to the disclosed technology ozone treatment devices may use filter media and various other suitable fan guards and filter components for any or none of the ozone generation and destruct inlets and outlets depending upon a desired configuration.

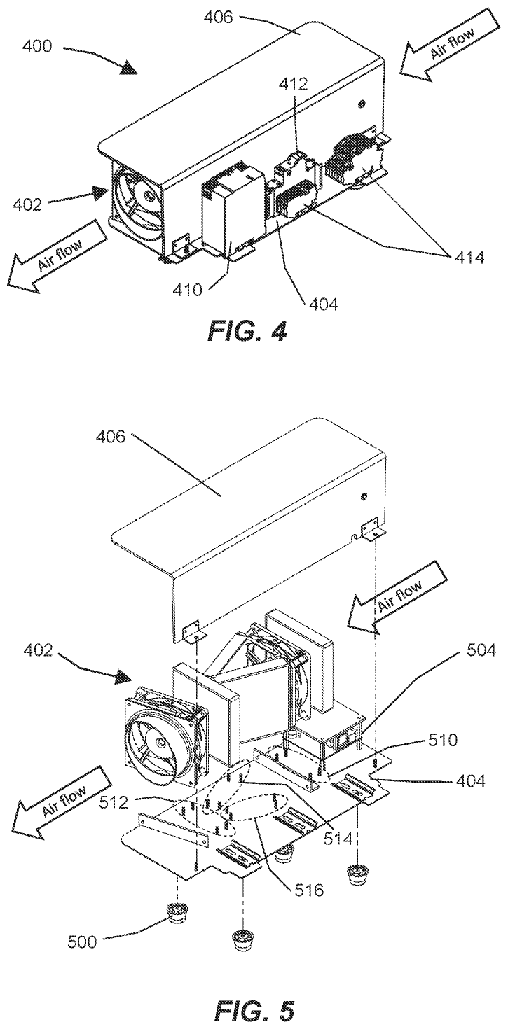

FIGS. 4-6 provide multiple views of an ozone destruct module 400 and included components according to various implementations. The ozone destruct module 400 is configured to be mounted between the ozone destruct inlet 120 and ozone destruct outlet 122 within the device housing 140 of FIG. 3. The ozone destruct module 400 is further configured to remove or destruct ozone as air moves through the destruct module along a destruct flow path. The destruct flow path extends through the destruct module 400 from right to left in the example depicted in FIGS. 4-6, as indicated by the arrows showing the direction of air flow.

Returning to FIG. 4, a perspective view of the ozone destruct module 400 is shown. According to the illustrated implementation, the destruct module 400 includes a destruct subassembly 402 mounted to a support plate 404. A cover 406 is mounted to the support plate 404 about the destruct subassembly 402. According to various implementations, the destruct module 400 also includes power electronics mounted to the support plate 404 for operating the destruct module 400 and, optionally, other components of an ozone treatment device. In the example of FIG. 4, the power electronics include a power supply 410, a circuit breaker 412, and terminal blocks 414 for connecting various components to the power supply and circuit breaker.

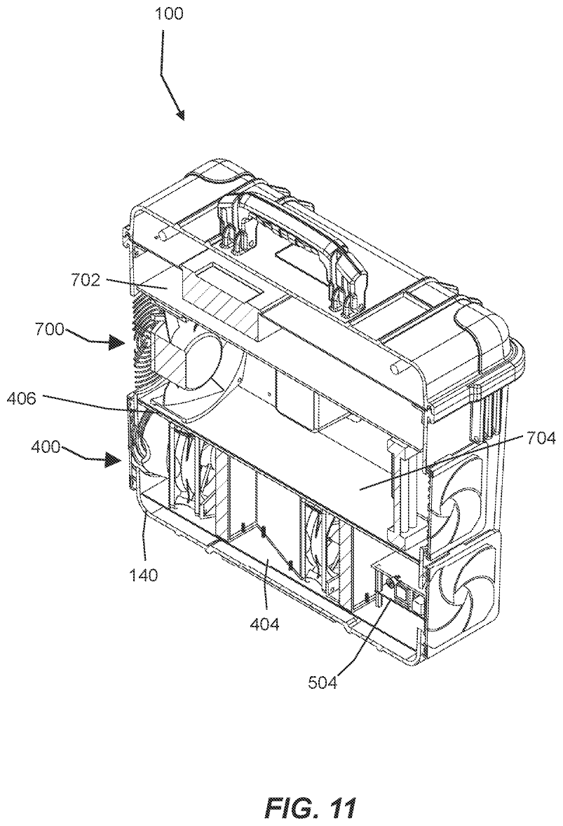

FIG. 5 is an exploded perspective view of the ozone destruct module 400 showing the assembly of the support plate 404, destruct subassembly 402 and cover 406. According to the illustrated implementation, a number of brackets, rails (e.g., DIN rails), and studs are attached to the support plate 404 for mounting components of the destruct subassembly 402. The destruct module 400 also includes feet 500 that mount to the bottom of the support plate for positioning the destruct module above the bottom of the housing as shown in the cross-sectional view of FIG. 11.

Continuing with reference to FIG. 5, the cover 406 in this implementation has two sides formed at a right angle. As shown in FIG. 4, one side separates the destruct subassembly 402 from the power electronics and the other side is positioned over the top of the destruct subassembly. According to some implementations the cover 406 is formed from a plastic, such as PVC, although any suitable material may be used. FIG. 5 also depicts an ozone sensor assembly 504 mounted to the support plate 404 according to various implementations. In this configuration the sensor assembly 504 is positioned at the beginning of the ozone destruct flow path before ozonated air enters the destruct subassembly 402.

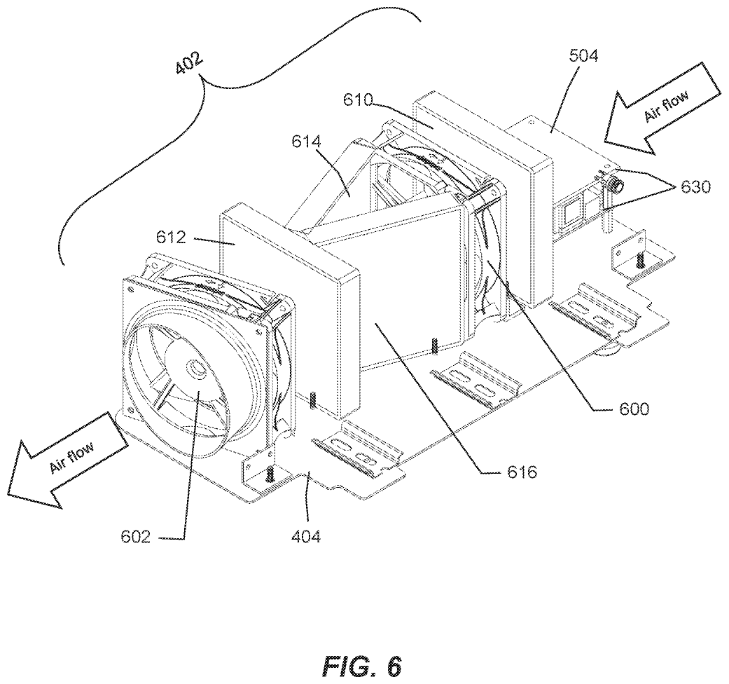

FIG. 6 is a perspective view of the ozone destruct subassembly 402 and ozone sensor assembly 504 mounted to the support plate 402 according to various implementations. In this example the ozone destruct subassembly 402 includes an intake fan 600, an output fan 602, and a number of ozone destruct filters mounted on the support plate 404. During operation, the intake and output fans 600, 602 bring ozone-filled air into the ozone destruct module through the ozone destruct inlet of the treatment device. The fans move the air along the destruct flow path by the ozone sensor assembly 504 and through the ozone destruct filters before venting it out through the ozone destruct outlet of the treatment device.

In the depicted example, the intake and output fans 600, 602 are electric fans rated for 70 cubic feet per minute (CFM). Fans capable of other speeds may also be used. Further, while two fans are illustrated in this example, fewer or greater than two fans may be used. Although not depicted in the figures, the fans are electrically coupled to the power supply 410 shown in FIG. 4 through, for example, one of the terminal blocks 414 and are operated by a controller as will be described further herein.

Returning to FIG. 6, the ozone destruct filters are configured as square-shaped, removable media panels that are held in position by pairs of studs mounted to the support plate 404. In the present configuration, the ozone destruct filters include an inlet filter 610 and an outlet filter 612 mounted transverse to the direction of air flow. The inlet filter 610 is removably held in place by a first group of studs 510 protruding from the support plate 404 as shown in FIG. 5. The outlet filter 612 is held in place in a similar manner by a second group of studs 512.

According to this implementation, the ozone destruct filters also include two intermediate filters 614, 616 angled toward one another between the intake fan 600 and the outlet destruct filter 612. The intermediate filters 614, 616 are held in place by third and fourth groups of studs 514, 516, respectively as shown in FIG. 5. In some cases positioning the intermediate filters 614, 616 at angles to the air flow direction allows for the use of more destruct filters than would otherwise fit within the destruct module, thus increasing the available surface area for ozone to react with the filter media. In addition, in some cases the angle of the intermediate destruct filters 614, 616 causes the air to become turbulent and flow throughout the entire square filter face instead of only a circular portion in the middle of the filter face.

According to various implementations, the ozone destruct filters are formed from a destruct media such as activated carbon. In some cases the destruct filters may have an ozone reduction rate of about 90%. Other suitable ozone destruction materials and forms may also be used. In addition, in some cases an optional seal member is attached around the perimeter of each destruct filter. The seal members can help create a more effective seal between the ozone destruct filters and the surrounding support plate 404, sides of the cover 406, and also a side of the treatment device housing in some cases. The seal members can thus help direct ozonated air through the ozone destruct filters and reduce the amount of air leaking around the perimeters of the filters. In some cases the seal member can be a length of weather stripping formed from foam, rubber, vinyl and/or other suitable materials.

Continuing with reference to FIG. 6, the ozone sensor assembly 504 in various implementations includes an ozone sensor and associated circuitry mounted to one or more circuit boards 630. As shown in FIG. 6 and also in FIG. 11, the circuit boards 630 are positioned above the support plate 404 at the beginning of the destruct flow path just before the inlet destruct filter 620. Other placements within the ozone destruct module 400 are also possible. According to certain implementations, the ozone sensor communicates with a controller as part of an ozone monitor that determines when the concentration of ozone in the air is above or below a certain threshold. For example, in some cases the sensor and/or controller determines whether the ozone concentration is at, above or below 0.1 parts per million (ppm). In certain implementations the ozone treatment device provides an indication (e.g., to an operator) when the ozone concentration is above or below the threshold. In certain implementations the treatment device controller may turn on an indicator light (e.g., LED) to show that the ozone concentration is above a particular threshold. In some cases the treatment device controller may turn on a different indicator light to show that the ozone concentration is below a particular threshold.

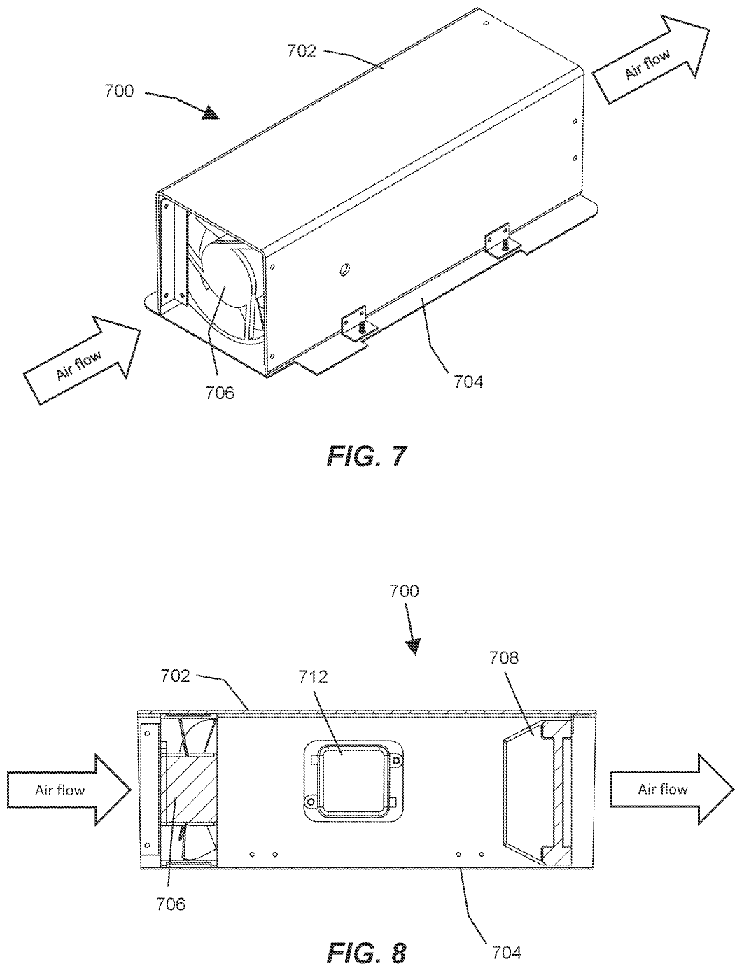

FIGS. 7-9 are views of an ozone generator 700 according to various implementations of the disclosed technology. The ozone generator 700 is generally configured to be mounted between the ozone generation inlet 110 and ozone generation outlet 112 within the device housing 140 of FIG. 3. The ozone generator 700 is further configured to generate and add ozone to air moving through the ozone generator along an ozone generation flow path. The generation flow path extends through the generator 700 from left to right in the example depicted in FIGS. 7-9, as indicated by the arrows showing the direction of air flow.

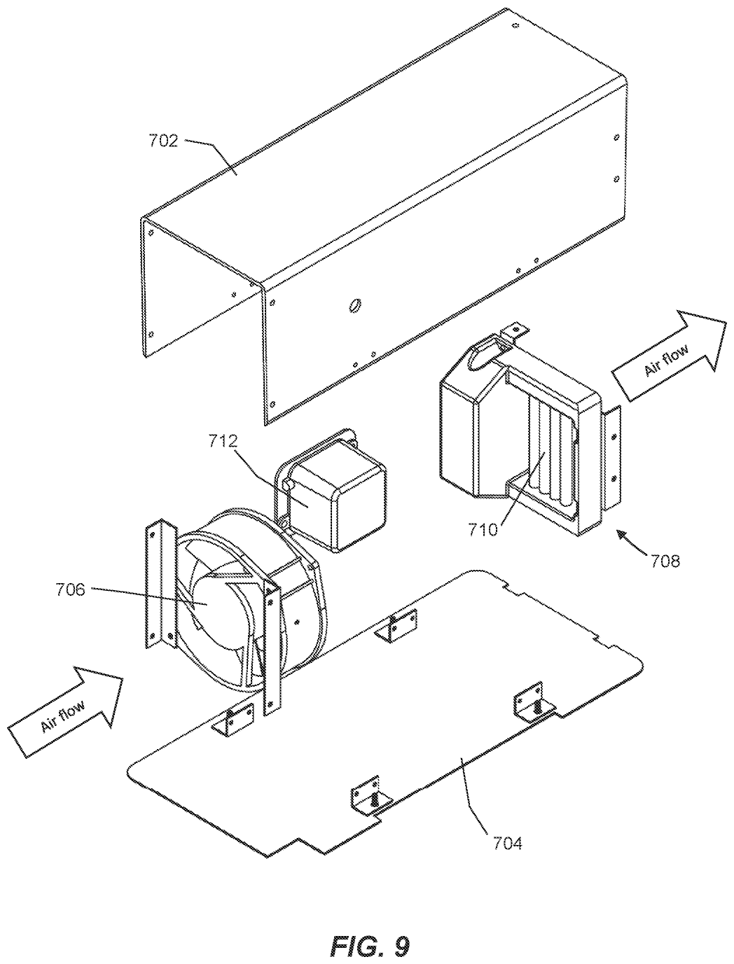

FIG. 7 is a perspective view, FIG. 8 provides a cross-sectional view, and FIG. 9 provides an exploded perspective view of the ozone generator 700 according to various implementations of the disclosed technology. The ozone generator 700 in this implementation includes a cover or tube 702 that is mounted to a support plate 704 to form a housing for the ozone generator 700. According to some implementations the tube 702 is formed from a plastic, such as PVC, although any suitable material may be used. An intake fan 706 is mounted at one end of the tube 702 and an ozone generating cell 708 is mounted near the opposite end of the tube 702. As shown in FIG. 9, in this example the ozone generating cell 708 includes a number of corona discharge tubes 710.

When mounted within the device housing, the arrangement shown in FIGS. 8 and 9 positions the intake fan 706 and the ozone generating cell 708 between the ozone generation inlet and outlet thereby defining the ozone generation flow path. According to this implementation, the fan 706 draws ambient air in through the ozone generation inlet and forces the air through the generator and out through the ozone generation outlet. As the air passes around and through the corona discharge tubes 710, the tubes 710 generate ozone from oxygen molecules in the air. It will be appreciated that this arrangement is but one possible configuration of the ozone generation flow path and that other configurations are also possible.

As shown in FIGS. 8-9, the ozone generator 700 includes a transformer 712 mounted to an inner side wall of the tube 702 in this implementation. The transformer 712 is configured to sufficiently transform the voltage from a power supply to operate the ozone generating cell 708. In various implementations the ozone generator 700 is powered from a common power supply shared with an ozone destruct module within an ozone treatment device. In the depicted example, the ozone generator 700 is configured to be electrically coupled with and supplied by the power supply 410 mounted to the ozone destruct module 400 illustrated in FIG. 4. In some cases the ozone generator 700 is electrically coupled to the power supply via one or more of the terminal blocks 414 and/or a device controller. As an example, an ozone treatment device controller can be configured to selectively turn the intake fan 706 and ozone generating cell 708 on and off by connecting and disconnecting power provided by the power supply 410.

The ozone generating cell in this and other implementations include corona discharge tubes 710, though other types of known, suitable ozone generating cells may be used. In the depicted example, the ozone generating cell 708 includes four corona discharge tubes 710. It will be appreciated that any suitable number of cells and/or discharge tubes may be used. The ozone generating cell(s) may be rated for generating any suitable amount of ozone depending upon the requirements of a particular implementation, including the sizes of rooms and ozone concentrations needed. In some cases the ozone generating cell is capable of generating ozone at a rate of about 6-10 g/hr.

The ozone generator fan 706 may have the same or different capabilities as the ozone destruct fans depending on the air speed and volume desired for a particular implementation. In some cases the generator fan 706 has a rated output of 120 CFM. Included power supply components are compatible with and provide the electrical power needed for operating the fan, ozone generating cell, and the controller. One example of a suitable power supply has a 120-230 V AC input and output rated at 24 V DC at 2.5 A.

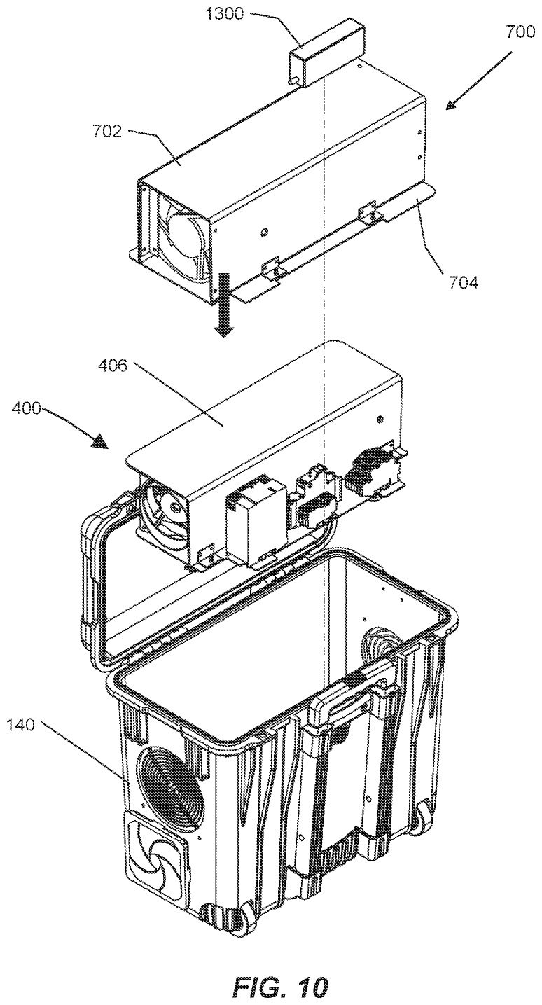

FIG. 10 is a perspective view illustrating the mounting of the ozone destruct module 400 of FIG. 4 and the ozone generator 700 of FIG. 7 within the housing 140 of FIG. 3 according to some implementations. FIG. 11 is a perspective cross-sectional view of the ozone treatment device 100 shown in FIG. 1 that illustrates the relative positions of the ozone generator 700 and the destruct module 400. In this example, the ozone generator 700 is positioned in a top portion of the housing 140 above the ozone destruct module 400. In some situations this relative mounting can increase access to the ozone generator 700, thereby making it easier to maintain and repair than if it was located underneath the destruct module 400.

Continuing with reference to FIGS. 10 and 11, in some cases the covers and/or support plates of the ozone generator 700 and destruct module 400 separate the ozone generation flow path and the ozone destruct flow path to prevent cross airflows that could impact the effectiveness of the device. For example, in some cases the cover 702 and support plate 704 of the generator combine to isolate the ozone generation flow path from the ozone destruct module 400 within the housing 140. In some cases the generator support plate 704 can also help reduce and/or prevent undesirable air flows between the ozone generator 700 and the ozone destruct module. For example, in certain implementations the generator support plate 704 and/or the tubular cover 702 substantially seal off the ozone generator flow path from the ozone destruct flow path. In certain implementations the side and top sections of the destruct module cover 406 provides another barrier to prevent air flows from crossing between the generator 700 and the destruct module 400.

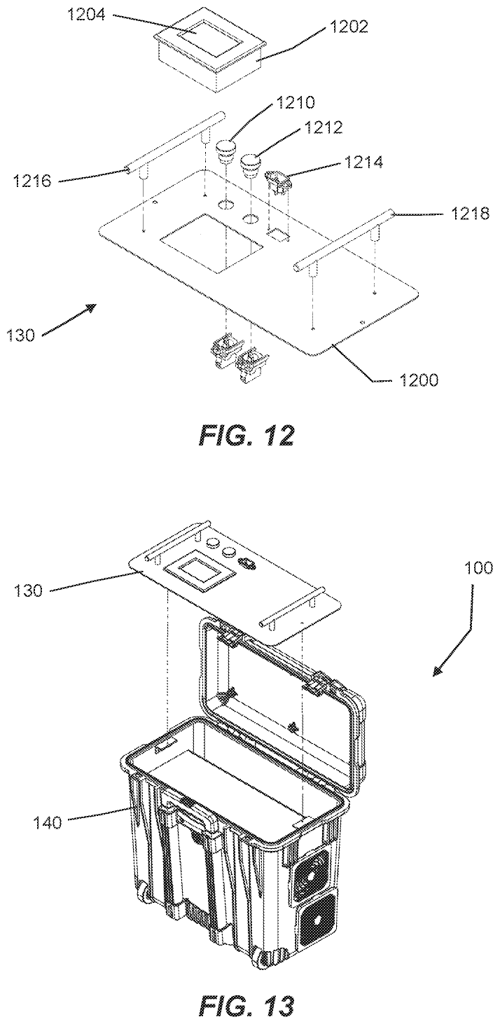

FIG. 12 is an exploded, perspective view of the control panel 130 for the ozone treatment device 100 shown in FIG. 1 according to certain implementations. FIG. 13 is an exploded, perspective view showing how the control panel 130 is mounted to the housing 140 of FIG. 3 in various implementations.

Returning to FIG. 12, the depicted control panel generally provides a human-machine interface (HMI) with controls and indicators mounted to a support plate 1200 for operating the ozone treatment device 100. In particular, the control panel 130 includes a controller 1202 mounted to the support plate 1200. The controller 1202 in this example both receives inputs from an operator and controls operation of the ozone generator 700 and the ozone destruction module 400. As an example, in this case the controller 1202 includes an input device 1204 in the form of a touch screen for receiving inputs from an operator. In some cases the input device may be separate from the controller.

According to some implementations, the controller is microprocessor-based or microcontroller-based. In some cases the controller includes a programmable logic controller (PLC) having programmable memory that stores instructions for configuring the PLC to carry out various operations. One example of a suitable controller that includes a PLC as well as a touch screen input device is the EZTouch mini.PLC® controller available from EZAutomation. Other types of controllers may also be used.

As shown in FIG. 12, the illustrated control panel 130 also includes first and second indicators 1210, 1212, a power plug receptacle 1214, and handles 1216, 1218 which can be useful for installing and removing the control panel to perform maintenance on the treatment device. In this implementation the first and second indicators 1210, 1212 are lights. For example, in some cases the first indicator 1210 is a red LED indicator and the second indicator 1212 is a green LED indicator. The power plug receptacle 1214 is configured to receive a plug end of a power cord. An internal power supply (e.g., power supply 410) is electrically coupled to the power plug receptacle 1214. In some cases a separate power supply 1300 (shown in FIG. 10) is coupled with the main power supply 410 or directly to the power plug receptacle 1214 for powering the controller 1202.

When fully assembled, the controller 1202 is electrically coupled to the power supplies, the fans, the ozone generating cells, and the indicators. Such connections enable the controller to control operation of the various components by, for example, turning power to individual components on and off. As an example, in some cases the controller 1202 is configured to turn on the first indicator 1210 and turn off the second indicator 1212 when an ozone concentration is above a particular threshold level. The controller may also be configured to turn off the first indicator 1210 and turn on the second indicator 1212 when the ozone concentration goes below the threshold level.

As previously noted, certain implementations of the disclosed technology provide a convenient and effective way to generate ozone to disinfect an enclosed space and then destruct the ozone in the space to allow people to reenter the space more quickly than might otherwise be possible. According to certain implementations the controller is configured to operate the ozone generator to generate ozone. In some cases the controller is configured to generate ozone for a certain time period in order to generate a particular concentration of ozone. In some cases other suitable factors and/or criteria may be used.

In some implementations the controller may be configured or programmed to produce a particular concentration of ozone for an enclosed space of a particular size in terms of volume. As an example, the controller may be configured to generate an ozone concentration of 5 ppm for an enclosed space with a particular volume. In some cases an operator picks or enters the volume of the enclosed space using an input device of the treatment device. In this type of implementation, the controller is configured to receive the input from the input device and determine the time needed to generate the desired concentration of ozone for the corresponding volume. The controller is configured to then generate ozone for the determined time period.

In certain implementations the controller calculates the time period needed to generate the desired ozone concentration for a space with a particular volume. In some cases the controller may determine the time period based on the following relationships:

$$\frac{amount of ozone}{volume} = concentration of ozone \qquad ( 1 )$$

$$amount of ozone = rate of production \times time period \qquad ( 2 )$$

As just one possible example, in some cases an ozone treatment device according to the disclosed technology can generate 6-10 grams of ozone per hour. The controller may be configured to determine the time needed to generate a concentration of 5 ppm ozone in a space of 1,000 cubic feet at this rate. In general, 2.14 mg of ozone will produce an ozone concentration of 1 ppm in 1 cubic meter of air:

$$\frac{2.14 mg {O}^{3}}{1 {m}^{3}} = 1 ppm {O}^{3}$$

Converting to grams ozone per cubic foot and multiplying by five yields an answer of 0.3 grams of ozone for generating a concentration of 5 ppm ozone in a 1,000 cubic foot volume.

As will be appreciated, in some cases this type of calculation generally provides an initial and/or theoretical amount of ozone needed for a particular concentration in a given space. In certain implementations, a larger amount of ozone may be needed to achieve the desired concentration. For example, in some cases as much as five times the initial calculated amount may be generated to achieve the desired ozone concentration. In some cases the controller is configured to produce five times as much ozone as an amount calculated using the above methodology.

Continuing with the example above, in some cases the controller is configured to generate 1.5 grams (e.g., 5×0.3 grams) of ozone to achieve a concentration of 5 ppm ozone in a 1,000 cubic foot space. At a rate of 6-10 grams of ozone per hour, it would likely take between about 9-15 minutes to generate the 5 ppm ozone throughout the 1,000 ft³ space.

According to certain implementations, the controller may use one or more additional or alternative methods to determine the time needed to generate the desired ozone calculation. As just one example, in some cases the controller could be configured to receive the room size from an input device and then use a look-up table to find the time needed to produce a desired concentration corresponding to the volume input. In some cases the controller is operably coupled with an ozone sensor, such as the ozone sensor assembly 504 shown in FIGS. 5-6. In these implementations, the controller can be configured to begin ozone production and then stop the generation of ozone when the ozone sensor determines that the desired concentration level has been achieved.

According to certain implementations the controller is configured to operate the ozone destruct module to destroy ozone remaining in an enclosed space. In some cases the controller is configured to operate the destruct unit for a certain time period in order to bring an ozone concentration down to a particular level. In some cases the controller is configured to calculate the associated destruct time period based on the size of the space, the concentration of ozone present, a reduction rate of the destruct media, and the air flow based on fan speed.

As an example, in some cases the ozone treatment device may at first generate a concentration of 5 ppm ozone within a 1,000 cubic foot room. After generating the ozone, the controller is configured to operate the destruct unit to reduce the concentration. Assuming a fan speed of 70 cubic feet per minute (CFM) and 100% reduction by the destruct media, the controller would determine that 1000/70=14.85 minutes are needed to cycle the air through the destruct media to reduce the ozone concentration. In another example, the destruct media may have a 90% reduction rate. In this case the controller would determine that a first period of 14.85 minutes would result in a 0.5 ppm concentration, and that a second cycle (in total about 30 minutes) would achieve a concentration of 0.05 ppm ozone.

According to certain implementations, the controller may use one or more additional or alternative methods to determine the time needed for operating the ozone destruct module. As an example, in some cases the controller could be configured to receive the room size from an input device and then use a look-up table to find the time needed to reduce the concentration to a desired level within the corresponding volume. In some cases the controller is operably coupled with an ozone sensor. In these implementations, the controller can be configured to continue operation of the ozone destruct module until the ozone sensor determines that the desired concentration level has been achieved.

According to various implementations, the treatment device is configured to generate a desired concentration of ozone and then automatically destruct the ozone to a satisfactorily low level based. In some cases the device controller receives a desired ozone treatment time from a user through an input device. In some cases the controller receives an estimated room size from the input device and then calculates or looks up a corresponding treatment time. The treatment device then generates ozone for the determined treatment time. In some cases the controller may then measure the ozone concentration using the ozone sensor assembly. In the case that the measured ozone concentration is insufficient, the treatment device can generate ozone for an additional amount of time and then repeat the measurement, and optionally additional ozone generation, as needed.

In various implementations the controller is configured to operate the ozone destruct module for a dissipation time that corresponds to the particular treatment time just completed. In some cases the controller may calculate a dissipation or destruction time based on the treatment time, the size of the treated space, and/or the desired concentration of ozone being generated. In various cases destruction times are preprogrammed to correspond to various treatment times so that the controller automatically operates the destruct module for the destruction time corresponding to the selected treatment time. Table 1 provides a list of treatment times and corresponding dissipation times for various treatment spaces according to various implementations:

TABLE 1

| | Minimum | | |
|---|---|---|---|
| Area to be | Treatment | | Minimum Ozone |
| Treated | Time | | Dissipation Time |
| 0-100 sq ft | 15 | minutes | 10 minutes |
| 100-250 sq ft | 30 | minutes | 15 minutes |
| 250-500 sq ft | 1 | hour | 15 minutes |
| 501-1000 sq ft | 2 | hours | 30 minutes |
| 1001-1500 sq ft | 4 | hours | 1 hour |
| 1501-2000 sq ft | 6-8 | hours | 1 hour |
| 2001-2500 sq ft | 8 | hours | 2 hours |
| 2501-3000 sq ft | 12 | hours | 2 hours |

According to various implementations, the treatment device controller is preprogrammed with a list of treatment times, such as the treatment times shown in Table 1. In such cases a user can select a treatment time corresponding to the size of the space to be treated. The treatment device then begins ozone generation for the selected treatment time, followed by operation of the ozone destruction module for the dissipation time corresponding to the selected treatment time. According to various implementations, the controller will measure the ozone concentration at the end of the dissipation time using the ozone sensor assembly. If the ozone concentration meets the desired level, the treatment device will stop operation. If the ozone concentration is higher than the desired level, the controller will continue operation of the destruct module for another amount of time. The ozone concentration can then be sampled again and ozone destruction can be continued as needed until the concentration is below the desired threshold.

In various implementations the ozone treatment device generates an ozone concentration sufficient to treat a space for a desired purpose such as, for example, to destroy airborne pathogens. In some cases the treatment device is configured to produce an ozone concentration of about 5 ppm. In some cases the treatment device is configured to produce a concentration of about 10 ppm. In various implementations the controller will signal via a first indicator (e.g., a red LED) that the ozone concentration within the space is above a threshold and therefore people should not enter the space. The controller can also signal via a second indicator (e.g., a green LED) that the ozone concentration is below the threshold and thus people may enter the space. In some cases the ozone concentration threshold for turning off a first indicator and turning on a second indicator is 0.1 ppm.

Although the disclosure has been described with reference to preferred embodiments, persons skilled in the art will recognize that changes may be made in form and detail without departing from the spirit and scope of the disclosed apparatus, systems and methods.

What is claimed is:

1. An ozone treatment device, comprising:
a housing comprising a first air inlet, a second air inlet, a first air outlet, and a second air outlet;
a controller mounted to the housing;
an ozone destruct module coupled to the controller and mounted within the housing between the first air inlet and the first air outlet; and
an ozone generator coupled to the controller and mounted within the housing between the second air inlet and second air outlet,
wherein the controller is configured to generate ozone with the ozone generator for a first time period, and
wherein the controller is configured to automatically destruct ozone with the ozone destruct module for a second time period following the first time period.

2. The ozone treatment device of claim 1, further comprising an input device coupled to the controller, wherein the controller is further configured to receive an operator input from the input device and determine the first time period based on the operator input.

3. The ozone treatment device of claim 2, wherein the operator input is a treatment time.

4. The ozone treatment device of claim 2, wherein the operator input is a size of a room.

5. The ozone treatment device of claim 2, wherein the controller is configured to determine the second time period based on the operator input.

6. The ozone treatment device of claim 5, wherein the ozone destruct module comprises destruct media and a fan, and wherein the controller is further configured to determine the second time period based on a reduction rate of the destruct media and a speed of the fan.

7. The ozone treatment device of claim 1, further comprising an ozone sensor.

8. The ozone treatment device of claim 1, wherein the ozone generator comprises a support plate and wherein the support plate is mounted in the housing above the ozone destruct module.

9. The ozone treatment device of claim 8, wherein the support plate substantially seals off the ozone generator from the ozone destruct module.

10. The ozone treatment device of claim 1, wherein the housing comprises a portable case comprising a handle for hand-carrying.

11. The ozone treatment device of claim 1, wherein the housing comprises a rollable housing comprising wheels.

12. An ozone treatment device, comprising:
a housing comprising an ozone generation flow path and an ozone destruct flow path substantially sealed off from the ozone generation flow path, wherein the ozone generation flow path and the ozone destruct flow path are vertically stacked in the housing, one over the other;
an ozone generator positioned in the ozone generation flow path;
an ozone destruct module positioned in the ozone destruct flow path;
an ozone sensor; and
a controller coupled to the ozone generator, the ozone destruct module, and the ozone sensor, the controller configured to generate ozone with the ozone generator for a first time period and then automatically destruct ozone with the ozone destruct module for a second time period following the first time period.

13. The ozone treatment device of claim 12, further comprising an input device coupled to the controller, wherein the controller is further configured to receive an operator input from the input device and determine the first time period and the second time period based on the operator input.

14. The ozone treatment device of claim 12, wherein following the first time period, the controller is configured to determine an ozone concentration level using the ozone sensor and, if the ozone concentration level does not meet a concentration threshold level, generate ozone with the ozone generator for an additional time period.

15. The ozone treatment device of claim 12, wherein following the second time period, the controller is configured to determine an ozone concentration level using the ozone sensor and, if the ozone concentration level does not meet a concentration threshold level, destruct ozone with the ozone destruct module for an additional time period.

16. A method for operating an ozone treatment device, comprising:
generating ozone with an ozone treatment device for a first time period, the ozone treatment device comprising an ozone generator, an ozone destruct module, and a controller coupled to the ozone generator and the ozone destruct module; and
automatically destructing ozone with the ozone treatment device for a second time period following the first time period.

17. The method of claim 16, further comprising receiving an operator input with an input device of the ozone treatment device and determining, with the controller, the first time period and the second time period based on the operator input.

18. The method of claim 17, wherein the operator input is a room size.

19. The method of claim 16, further comprising determining, with the controller, an ozone concentration level sensed by an ozone sensor of the ozone treatment device and, if the ozone concentration level does not meet a concentration threshold level, generating ozone with the ozone treatment device for an additional time period.

20. The method of claim 16, further comprising determining, with the controller, an ozone concentration level sensed by an ozone sensor of the ozone treatment device and, if the ozone concentration level does not meet a concentration threshold level, destructing ozone with the ozone destruct module for an additional time period.
